(19) United States
(12) Patent Application Publication
(10) Pub. No.: US 2003/0167158 A1
(43) Pub. Date: Sep. 4, 2003

(54) Font for displaying genetic information

(76) Inventor: Brian Seed, Boston, MA (US)

Correspondence Address: HALE AND DORR, LLP, 60 STATE STREET, BOSTON, MA, 02109

(21) Appl. No.: 10/116,916

(22) Filed: Apr. 5, 2002

Related U.S. Application Data
(60) Provisional application No. 60/282,022, filed on Apr. 6, 2001.

(51) Int. Cl. G06G 7/48; G06G 7/58; G06F 19/00; G01N 33/48; G01N 33/50

(52) U.S. Cl. 703/11; 702/20

(57) ABSTRACT

The invention provides fonts for displaying genetic information. Each font comprises a set of glyphs, wherein each glyph of the set comprises a first alphanumerical character positioned vertically above a second alphanumerical character, wherein the first character represents a first nitrogenous base and the second character represents a second nitrogenous base that is complementary to the first nitrogenous base, and wherein each glyph of the set is displayed in response to an entered command, the entered command assigned to the displayed glyph.

Also provided are methods for displaying genetic information using a font of the invention; methods for displaying a double stranded codon and an amino acid encoded thereby using a font of the invention; and computers and computer program products that use the font of the invention for displaying genetic information.

### CROSS REFERENCE TO RELATED APPLICATIONS

This application claims benefit from U.S. provisional patent application No. 60/282,022, filed Apr. 6, 2001, the entire text of which is hereby incorporated by reference.

### BACKGROUND OF THE INVENTION

1. Field of the Invention

This invention relates to a font, particularly a font for use on an editor.

2. Summary of the Related Art

Recent efforts to sequence the human genome and analyze the approximately 30,000 genes in human DNA (see, e.g., http://www.ornl.gov/hgmis/) have made it critically important to be able to quickly and accurately manipulate genetic information.

Currently, genetic information, as contained within nucleic acid molecules, is schematically displayed and manipulated on standard nucleic acid editing programs on a word processor or a computer. These editing programs enable analysis of nucleic acid molecules, such as restriction endonuclease mapping, to facilitate further manipulation and use of the nucleic acid molecule.

Various nucleic acid editing programs are known, including DNAstrider, MacPlasmap, and a number of different GCG programs (Wisconsin Sequence Analysis Package Program, Genetics Computer Group, Inc., Madison, Wis.). Typically (e.g., in GCG programs), a double stranded nucleic acid molecule (e.g., DNA) is displayed as two rows of letters (representing nitrogenous bases) representing the complementary strands of nitrogenous bases, with a single line separating the rows. Where the nucleic acid molecule encodes a protein, the single letter amino acid code is displayed below its codon in the nucleic acid molecule.

For example, a typical sequence generated by the Map program (a GCG program) is as follows, where the single amino acid letter code appears below the first nitrogenous base of the 3-base codon encoding the amino acid and where the restriction endonuclease recognition sites are above the double stranded nucleic acid molecule:

| | | |
|---|---|---|
| | HgaI | |
| | SimI | |
| | NlaIII\| | |
| | BsaJI \|\| | |
| | DsaI \|\| | |
| | NcoI \|\| | |
| | StyI \|\| | |
| | BsaHI \| \|\| RleAI | |
| | BspGI \| \| \|\| MnlI BseRI \| | |
| | BfaI \| \| \| \|\| MnlI \| SmaI CviJI \| \| CviJI | |
| | \| \| \| \| \|\| \| \| \| \| \| \| \| | |
| | GCTCCTAGTCCAGACGCCATGGGTCATTTCACAGCCCGGGACAAGGCTACTATCACAAGC | |
| 61 | ---------+---------+---------+---------+---------+---------+ | 120 |
| | CGAGGATCAGGTCTGCGGTACCCAGTAAAGTGTCGGGCCCTGTTCCGATGATAGTGTTCG | |
| | A P S P D A M G H F T P G D K A T I T S | - |

Routine molecular biology techniques involve the digestion of a nucleic acid molecule with a restriction endonuclease which cuts at a specific recognition site in the molecule. For example, the above sequence may be digested with the restriction endonuclease, SmaI, which cuts at the following site in a double stranded nucleic acid molecule:

```
                            ↓
5′ . . . C C C G G G . . . 3′
                  -----------
3′ . . . G G G C C C . . . 5′
                              ↑
```

resulting in the above nucleic acid molecule being cut into two segments as follows:

. . . GCTCCTAGTCCAGACGCCATGGGTCATTTCACAGCCC
---------+---------+---------+-------
. . . CGAGGATCAGGTCTGCGGTACCCAGTAAAGTGTCGGG
and
GGGACAAGGCTACTATCACAAGC . . .
--+---------+---------+
CCCTGTTCCGATGATAGTGTTCG . . .

Each of these two segments can then be ligated to another blunt end-cut nucleic acid molecule (e.g., digested with a blunt end cutting restriction endonuclease such as SmaI, or digested with a sticky-end cutting restriction endonuclease, where either the resulting sticky end is filled in using, e.g., DNA polymerase, or the overhang is removed using, e.g., a DNA exonuclease) to form a blunt end new nucleic acid molecule.

However, because all of the known fonts (e.g., Courier or Monaco) are one character in height, it is often difficult to readily cut and paste segments of a double stranded nucleic acid molecule using these fonts. For example, to represent cutting the above-described sequence at the SmaI, the user of the editor has to cut and paste each line. Were the user to simply scroll down after the SmaI site in the above sequence, the following sequence would be obtained:

| | | |
|---|---|---|
| | GGGACAAGGCTACTATCACAAGC | |
| 61 | ---------+---------+---------+---------+---------+---------+ | 120 |
| | CGAGGATCAGGTCTGCGGTACCCAGTAAAGTGTCGGGCCCTGTTCCGATGATAGTGTTCG | |

While it may appear to be a simple matter to remove the 3′ overhang, to obtain:

GGGACAAGGCTACTATCACAAGC
--+---------+---------+
CCCTGTTCCGATGATAGTGTTCG

in fact, when the user attempts to paste this blunt-ended sequence onto another blunt-ended sequence, such as:

. . . GCTCCTAGTCCAGACGCCATGGGTCATTTCACAGCCC
---------+---------+---------+-------
. . . CGAGGATCAGGTCTGCGGTACCCAGTAAAGTGTCGGG

the following sequence is obtained:

```
        ... GCTCCTAGTCCAGACGCCATGGGTCATTTCACAGCCC
GGGACAAGGCTACTATCACAAGC
                                                                                    --+---------+---------+
                                                                                    CCCTGTTCCGATGATAGTGTTCG
                ---------+---------+---------+-------
        ... CGAGGATCAGGTCTGCGGTACCCAGTAAAGTGTCGGG
```

Only by repeated cutting an pasting can the user of a standard DNA editing program obtain the correct sequence:

GCTCCTAGTCCAGACGCCATGGGTCATTTCACAGCCCGGGACAAGGCTACTATCACAAGC
---------+---------+---------+---------+---------+---------+
CGAGGATCAGGTCTGCGGTACCCAGTAAAGTGTCGGGCCCTGTTCCGATGATAGTGTTCG

This cutting and pasting routine is, of course, even more difficult with longer sequences and where more than two nucleic acid molecule fragments are pasted together to form a new nucleic acid molecule. Not only is the cutting and pasting routine, tedious and time-consuming, but, more importantly, cutting and pasting can also result in mistakes, such as including or deleting a nitrogenous base. Such additions or deletions not only affect the editor's ability to restriction endonuclease map the newly generated nucleic acid molecule, but also affect the editor's ability to correctly translate the newly generated nucleic acid molecule into protein, since an addition or deletion of a nitrogenous base will result in a frame shift, thereby altering the amino acid sequence of the encoded protein.

Thus, there exists a need to develop a font to facilitate the display and manipulation of genetic information.

### SUMMARY OF THE INVENTION

The present inventor has devised a genetic font that facilitates display, manipulation, and editing of genetic information on an editor. The invention provides a font for displaying, manipulating, and editing genetic information, as well as using the font of the invention for displaying a nucleic acid base pair and for displaying a double-stranded codon and an amino acid encoded thereby.

Accordingly, in a first aspect, the invention provides a font for displaying and editing genetic information comprising a set of glyphs, wherein each glyph of the set comprises a first alphanumerical character positioned vertically above a second alphanumerical character, wherein the first character represents a first nitrogenous base and the second character represents a second nitrogenous base that is complementary to the first nitrogenous base, and wherein each glyph of the set is displayed in response to an entered command, the entered command assigned to the displayed glyph.

In some embodiments of the first aspect of the invention, each glyph of the set occupies the same width. In certain embodiments, the first alphanumerical character is separated from the second alphanumerical character by a horizontal line. In certain embodiments, the first alphanumerical character and the second alphanumerical character are alphabetical letters. For example, the first alphanumerical character and the second alphanumerical character may be lower case alphabetical letters.

In certain embodiments of the first aspect, the font further comprises a first subset of glyphs, wherein each glyph of the first subset of the font comprises an alphanumerical character or a * symbol. In some embodiments, the alphabetical letter character of the first subset of the glyph is an upper case alphabetical letter. In various embodiments, the glyph of the first subset is positioned either above or below a second alphanumerical character of a glyph of the set of the font.

In certain embodiments, the font of the first aspect of the invention further comprises a second subset of glyphs, wherein each glyph of the second subset is an alphanumerical character.

In a second aspect, the invention provides a method for displaying genetic information represented by a set of glyphs, wherein each glyph of the set comprises a first alphanumerical character positioned vertically above a second alphanumerical character, wherein the first alphanumerical character represents a first nitrogenous base and the second alphanumerical character represents a second nitrogenous base that is complementary to the first nitrogenous base. The method of the second aspect comprises receiving an entered command to display one of the set of glyphs; identifying the glyph of the set assigned to the entered command; and displaying the identified glyph.

In a third aspect, the invention provides a computer program product in a computer-readable media for displaying genetic information represented by a set of glyphs, wherein each glyph of the set comprises a first alphanumerical character positioned vertically above a second alphanumerical character, wherein the first alphanumerical character represents a first nitrogenous base and the second alphanumerical character represents a second nitrogenous base that is complementary to the first nitrogenous base. In accordance with this aspect of the invention, the computer program product comprises means for receiving an entered command to display one of the set of glyphs; means for identifying the glyph of the set assigned to the entered command; and means for displaying the identified glyph.

In a fourth aspect, the invention provides a computer comprising at least one processor; memory associated with the at least one processor; a display; and a program supported in the memory for displaying genetic information represented by a set of glyphs, wherein each glyph of the set comprises a first alphanumerical character positioned vertically above a second alphanumerical character, wherein the first alphanumerical character represents a first nitrogenous base and the second alphanumerical character represents a second nitrogenous base that is complementary to the first nitrogenous base. According to this aspect of the invention, the program comprises means for receiving an entered command to display one of the set of glyphs; means for identifying the glyph of the set assigned to the entered command; and means for displaying the identified glyph.

In some embodiments of the second, third, and fourth aspects of the invention, the command is entered using a standard keyboard. In some embodiments, the glyph is displayed on a display screen with a cursor at a location on the screen, the location being the location of the cursor when the command is entered.

In a fifth aspect, the invention provides a method for displaying a double-stranded codon and an amino acid encoded by the codon, wherein the method comprises receiving an entered first command, second command, and third command, wherein each of the first command, second command, and third command, is assigned to a glyph of a first set of glyphs, wherein the glyph of the first set comprises a first alphanumerical character positioned vertically above a second alphanumerical character, wherein the first alphanumerical character represents a first nitrogenous base and the second alphanumerical character represents a second nitrogenous base that is complementary to the first nitrogenous base; receiving an entered fourth command, wherein the fourth command is assigned to a glyph of a second set of glyphs, wherein the glyph of the second set comprises a third alphanumerical character positioned vertically above or below a second character of a glyph displayed by any one of the first command, the second command, or the third command; identifying the glyph assigned to each of the entered first command, second command, third command, and fourth command; and displaying the identified glyphs.

In a sixth aspect, the invention provides a computer program product in a computer-readable media for displaying a double-stranded codon and an amino acid encoded by the codon, the computer program product comprising means for receiving an entered first command, second command, and third command, wherein each of the first command, second command, and third command, command is assigned to a glyph of a first set of glyphs, wherein the glyph of the first set comprises a first alphanumerical character positioned vertically above a second alphanumerical character, wherein the first alphanumerical character represents a first nitrogenous base and the second alphanumerical character represents a second nitrogenous base that is complementary to the first nitrogenous base; means for receiving an entered fourth command, wherein the fourth command is assigned to a glyph of a second set of glyphs, wherein the glyph of the second set comprises a third alphanumerical character positioned vertically above or below a second character of a glyph displayed by any one of the first command, the second command, or the third command; means for identifying the glyph assigned to each of the entered first command, second command, third command, and fourth command; and means for displaying the identified glyphs.

In a seventh aspect, the invention provides a computer comprising at least one processor; memory associated with the at least one processor; a display; and a program supported in the memory for displaying genetic information represented by a set of glyphs, wherein each glyph of the set comprises a first alphanumerical character positioned vertically above a second alphanumerical character, wherein the first alphanumerical character represents a first nitrogenous base and the second alphanumerical character represents a second nitrogenous base that is complementary to the first nitrogenous base. In accordance with this aspect of the invention, the program comprises means for receiving an entered first command, second command, and third command, wherein each of the first command, second command, and third command, command is assigned to a glyph of a first set of glyphs, wherein the glyph of the first set comprises a first alphanumerical character positioned vertically above a second alphanumerical character, wherein the first alphanumerical character represents a first nitrogenous base and the second alphanumerical character represents a second nitrogenous base that is complementary to the first nitrogenous base; means for receiving an entered fourth command, wherein the fourth command is assigned to a glyph of a second set of glyphs, wherein the glyph of the second set comprises a third alphanumerical character positioned vertically above or below a second character of a glyph displayed by any one of the first command, the second command, or the third command; means for identifying the glyph assigned to each of the entered first command, second command, third command, and fourth command; and means for displaying the identified glyphs.

In certain embodiments of the fifth, sixth, and seventh aspects of the invention, the fourth command is entered after each of the first command, the second command, and the third command is entered. In other embodiments, the fourth command is entered before at least one of the first command, the second command, and the third command is entered.

In another aspect, the invention features a method for displaying genetic information in a computer system, the computer system including a monitor and a keyboard. The method comprises defining a plurality of glyphs, each glyph including at least a first character and a second character, the first and second characters in each of the glyphs representing first and second complementary nitrogenous bases, respectively; defining a plurality of commands that may be entered into the computer system, each command being entered into the computer system by one or more keystrokes of the keyboard; establishing a correspondence between the commands and the glyphs, each of the commands corresponding to one of the glyphs, each command being associated with a first number and a second number, the first number being the number of keystrokes used to enter the command into the computer system, the second number being the number of characters in the glyph corresponding to the command, the second number being greater than the first number; and, in response to a first one of the commands being entered into the computer system, displaying the glyph corresponding to the first command on the computer monitor. In some embodiments, the method further comprises, in response to a second one of the commands being entered into the computer system, displaying the glyph corresponding to the second command on the computer monitor to the right of and adjacent to the glyph corresponding to the first command.

In another aspect, the invention provides method for displaying information in a computer system, the computer system including a monitor and a keyboard. In this aspect, the method comprises displaying two or more adjacent glyphs on the monitor, each glyph including at least a first character and a second character, the first and second characters in each of the glyphs representing first and second complementary nitrogenous bases, respectively; and defining a select command that may be entered into the computer system, the select command permitting simultaneous selection of all characters in one of the displayed adjacent glyphs without also selecting characters in any other of the displayed adjacent glyphs.

In certain embodiments, the select command further permitting selection of two or more adjacent glyphs displayed on the monitor. In some embodiments, the method further comprises defining a delete command that may be entered into the computer system, the delete command removing all selected glyphs from the monitor. In certain embodiments, a left glyph being a previously displayed glyph to the left of and adjacent to the selected glyphs, a right glyph being a previously displayed glyph to the right of and adjacent to the selected glyphs, the delete command further comprising moving the right glyph to the left so that it is adjacent to the left glyph. In certain embodiments, a right group of glyphs including the right glyph and all previously displayed glyphs to the right of the right glyph, the delete command further comprising moving the right group of glyphs to the left.

In some embodiments, the method further comprises defining a copy command that may be entered into the computer system, the copy command copying the selected glyphs into a buffer. In certain embodiments, the method further comprises defining a paste command that may be entered into the computer system, a left end glyph being the glyph at a left end of the selected glyphs, a right end glyph being the glyph at a right end of the selected glyphs, a right glyph being a previously displayed glyph, a left glyph being a previously displayed glyph to the left of and adjacent to the right glyph, the paste command displaying the glyphs in the buffer on the monitor such that the right end glyph is to the left of and adjacent to the right glyph, and such that the left end glyph is to the right of and adjacent to the left glyph.

In another aspect, the invention provides a method for displaying information in a computer system, the computer system including a monitor and a keyboard. According to this aspect, the method comprises defining a plurality of glyphs, each glyph including at least a first character and a second character, the first and second characters in each of the glyphs representing first and second complementary nitrogenous bases, respectively; defining a plurality of commands that may be entered into the computer system, each command being entered into the computer system by applying one or more keystrokes to the keyboard; establishing a correspondence between the commands and the glyphs, each of the commands corresponding to one of the glyphs; establishing a present cursor location on the monitor; in response to one of the commands being entered into the computer system, displaying the glyph corresponding to that command on the monitor at the present cursor location and then moving the present cursor location to the right of the glyph corresponding to that command; repeating the previous step in response to additional commands being entered into the computer; and defining a select command that may be entered into the computer system, the select command permitting simultaneous selection of all characters in a single glyph without also selecting characters in any other displayed glyphs adjacent to the single glyph.

### DETAILED DESCRIPTION OF THE PREFERRED EMBODIMENTS

The present inventor has devised a font for displaying and editing of genetic information on an editor, such as an editor on a word processor or a computer. By “editor” is meant a program that permits the user to create or modify data (as text or graphics) on a display screen. Preferably, the editor is a standard nucleic acid molecule editor including, without limitation, DNAstrider, MacPlasmap, and a number of different GCG programs (Wisconsin Sequence Analysis Package Program, Genetics Computer Group, Inc., Madison, Wis.).

Accordingly, in one aspect, the invention provides a font for displaying and editing genetic information comprising a set of glyphs, wherein each glyph of the set comprises a first alphanumerical character positioned vertically above a second alphanumerical character. In the font of the invention, the first alphanumerical character represents a first nitrogenous base and the second alphanumerical character represents a second nitrogenous base that is complementary to the first nitrogenous base. Each glyph of the set of the font of the invention is displayed in response to an entered command, the entered command assigned to the displayed glyph.

The font of the invention and the methods for using the font of the invention are preferably implemented in a general purpose computer. A representative computer is a personal computer or workstation platform that is, e.g., Intel Pentium®, PowerPC® or RISC based, and includes an operating system such as Windows®, OS/2®, Unix or the like. As is well known, such machines include a display interface (a graphical user interface or “GUI”) and associated input devices (e.g., a keyboard or a mouse).

The font of the invention (and method for using the font) is preferably implemented in software, and accordingly one of the preferred implementations of the invention is as a set of instructions (program code) in a code module resident in the random access memory of the computer. Until required by the computer, the set of instructions may be stored in another computer memory, e.g., in a hard disk drive, or in a removable memory such as an optical disk (for eventual use in a CD ROM) or floppy disk (for eventual use in a floppy disk drive), or downloaded via the Internet or some other computer network. In addition, although the various methods described are conveniently implemented in a general purpose computer selectively activated or reconfigured by software, one of ordinary skill in the art would also recognize that such methods may be carried out in hardware, in firmware, or in more specialized apparatus constructed to perform the specified method steps.

By “genetic information” is meant the nucleotide (i.e., nitrogenous base) sequence of a nucleic acid molecule. Of course, based on the genetic information, the ordinarily skilled biologist or bioinformaticist can easily determine the amino acid sequence of the protein encoded by the nucleic acid molecule by using the genetic code. Further, the ordinarily skilled biologist or bioinformaticist can manipulate the nucleic acid sequence of the nucleic acid molecule to introduce a different nitrogenous base (e.g., to create the recognition site of a restriction endonuclease) without altering the amino acid sequence of the encoded protein.

In accordance with the invention, by “glyph” is meant a symbol included in a font. By “character” is meant a symbol representing a nitrogenous base or an amino acid. By “nitrogenous base” is meant a nitrogenous base in a nucleic acid molecule. Included in this definition are nitrogenous bases bonded to other molecular structures, such as a nitrogenous base bonded to a sugar, such as deoxyribose, to form a nucleoside, and a nitrogenous base bonded to a sugar and a phosphate group to form a nucleotide.

By “complementary” is meant that a first nitrogenous base can form a Watson-Crick hydrogen bond base pair with a second nitrogenous base. For example, where the first nitrogenous base is adenine, the second nitrogenous base is either uracil or thymine, each of which is complementary to adenine. Likewise, where the first nitrogenous base is cytosine, the second nitrogenous base is guanine, which is complementary to adenine.

As used herein, by “adenine” is meant an adenine nitrogenous base. As used herein, an adenine may be unbonded to another molecule, or may be bonded to another molecule to form a larger molecule, such as an adenine coupled to a deoxyribose molecule to form deoxyadenosine (i.e., a nucleoside) or an adenine that forms a nucleotide in a nucleic acid molecule (e.g., deoxyadenylate in DNA).

As used herein, by “guanine” is meant a guanine nitrogenous base. As used herein, a guanine may be unbonded to another molecule, or may be bonded to another molecule to form a larger molecule, such as a guanine coupled to a deoxyribose molecule to form deoxyguanosine (i.e., a nucleoside) or a guanine that forms a nucleotide in a nucleic acid molecule (e.g., deoxyguanylate in DNA).

As used herein, by “thymine” is meant a thymine nitrogenous base. As used herein, a thymine may be unbonded to another molecule, or may be bonded to another molecule to form a larger molecule, such as a thymine coupled to a deoxyribose molecule to form deoxythymidine (i.e., a nucleoside) or a thymine that forms a nucleotide in a nucleic acid molecule (e.g., deoxythymidylate in DNA).

As used herein, by “cytosine” is meant a cytosine nitrogenous base. As used herein, a cytosine may be unbonded to another molecule, or may be bonded to another molecule to form a larger molecule, such as a cytosine coupled to a deoxyribose molecule to form deoxycytidine (i.e., a nucleoside) or a cytosine that forms a nucleotide in a nucleic acid molecule (e.g., deoxycytidylate in DNA).

In accordance with the invention, by “uracil” is meant a uracil nitrogenous base. As used herein, a thymine may be unbonded to another molecule, or may be bonded to another molecule to form a larger molecule, such as a uracil coupled to a ribose molecule to form uridine (i.e., a nucleoside) or a uracil that forms a nucleotide in a nucleic acid molecule (e.g., uridylate in RNA).

By “purine” is meant a nitrogenous base derived from a purine ring, wherein the purine ring has the structure:

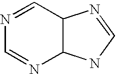

Non-limiting examples of pyrimidine nitrogenous bases include adenine and guanine, as defined herein.

By “pyrimidine” is meant a nitrogenous base derived from a pyrimidine ring, wherein the pyrimidine ring has the structure:

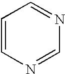

Non-limiting examples of pyrimidine nitrogenous bases include thymine, cytosine, and uracil, as defined herein.

By “keto” is meant guanine or thymidine, as defined herein.

By “amino” is meant adenine or cytosine, as defined herein.

By “weak” is meant a nitrogenous base that forms two hydrogen bonds with its complementary base. Thus, “weak” includes adenine or thymidine, as defined herein.

By “strong” is meant a nitrogenous base that forms three hydrogen bonds with its complementary base. Thus “strong” includes cytosine and guanine, as defined herein.

In accordance with the invention, “nucleic acid molecule” as used herein, means any chain of two or more nitrogenous bases that form a nucleic acid, preferably deoxyribonucleic acid (DNA) or ribonucleic acid (RNA), including, without limitation, complementary DNA (cDNA), genomic DNA, RNA, hnRNA, messenger RNA (mRNA), DNA/RNA hybrids, or synthetic nucleic acids (e.g., an oligonucleotide) comprising ribonucleic and/or deoxyribonucleic acids or synthetic variants thereof. The nucleic acid molecule of the invention includes, without limitation, an oligonucleotide or a polynucleotide. The nucleic acid molecule can be single stranded, or partially or completely double stranded (duplex). Duplex nucleic acid molecules can be homoduplex or heteroduplex.

In accordance with the invention, when an editor is used, each command entered is assigned a particular glyph, where the glyph is one character vertically positioned above another character. By “command” is meant the entering command to a computer, for example, by typing a keystroke or speaking using a voice activated computer program. In some preferred embodiments, the command is made by entering a keystroke using a standard keyboard. When the font of the invention is used, the glyph of the set of the font preferably appears where the cursor is located.

Thus, for purely exemplary purpose, and without limiting the font of the invention in any way, a command “t” may encode for the glyph:

“t”.
a

Once the “t” command is entered, the cursor appears after (i.e., to the immediate right of) the

“t”
a

glyph, and the next command, which may also be a “t”, is entered, and the second glyph assigned to the second “t” command appears immediately to the right of the first glyph (or, if the line has wrapped at the end of the page or screen, to the first position on the following line). Thus, in this example, the commands, “tt” will result in the following two glyphs in the font according to the invention:

“tt ”.
aa

The set of the glyphs of the font of the invention may include, without limitation, the following glyphs, each of which may be assigned to any command:

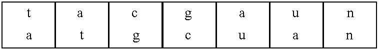

It will be appreciated any alphanumerical, diagrammatic, or iconic may be used as a character in a glyph of the invention. Where the symbol is alphanumerical, the alphanumerical language need not be a Romance language-based language, and may be, for example Arabic, Greek, English, or Braille.

In preferred embodiments, each glyph of the set of the font of the invention occupies the same width.

For example, both the glyph

“a”
t

and the glyph

“ C”
G

occupy the same width when displayed on either a screen of an editor (e.g., a computer screen) or on a printed page.

In preferred embodiments, each glyph of the set of the font of the invention occupies the same height.

For example, both the glyph

“c”
g

and the glyph

“ C”
G

occupy the same height when displayed on either a screen of an editor (e.g., a computer screen) or on a printed page.

In preferred embodiments of the invention, the first character is separated from the second character by a horizontal line. According to this embodiment, some non-limiting glyphs include:

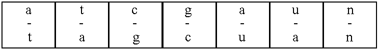

In various embodiments, the first and second alphanumerical characters of the set of the font are each an alphabetical letter. In some preferred embodiments, the first and second characters are each a lower case alphabetical letter.

In certain embodiments, the font of the invention further comprises a first subset of glyphs, wherein each glyph of the first subset comprises a third character which is positioned above or below a second character of a glyph of the set of the font. In certain embodiments, the third character is an alphabetical letter or a * symbol. In some preferred embodiments, the alphabetical letter is an upper case alphabetical letter.

Thus, one non-limiting variation of the embodiment in which the glyph of the first subset is positioned below a second character of a glyph of the set, the second character of the preceding glyph of the set of the font is positioned directly above the third character of the subsequent glyph. In this embodiment, the glyph of the subset (i.e., the third character) is displayed vertically below the second character of the preceding glyph, where the preceding glyph comprises a second character positioned vertically below a first character. Thus, where the glyph of the subset of the font of the invention is displayed vertically below the second character of a preceding glyph of the set of the font, the glyph of the subset appears to the left of the cursor on the computer screen below the second character of the preceding glyph. It should be noted that at the location of the cursor, a command entering a glyph of the subset of the invention occupies zero-width (i.e., the cursor remains where it is after the command has been entered), while the glyph of the subset occupies the same width of the preceding glyph of the set of the font and is positioned vertically below the preceding glyph.

In a non-limiting example, the command “t” is assigned to a glyph of the set of the font, namely,

```
“t”,
  —
  a
```

and the command “M” is assigned to the glyph, “M” of the subset of the font that is displayed vertically below the second character of the preceding glyph. In this example, entry of the command, “t” results in the display of the glyph

```
“t”,
  —
  a
```

where the cursor appears immediately to the right of the glyph.

The next command, “M” is assigned to the glyph, “M” of the subset of the font, and is entered immediately after entry of the “t” command. Since the “M” command has zero-width, when “M” is entered, the cursor remains after the

```
“t”
    —
    a
```

glyph.

In this non-limiting example of this embodiment of the font of the invention the commands, “tM” would result in the glyphs,

```
“t”.
    —
    a
    M
```

Continuing this non-limiting example, if the commands “tMt” were entered, the following glyphs are generated:

```
“tt”.
  --
  aa
  M
```

Note that if the lines in which the

```
“t”
  -
  a
  M
```

glyphs are located was filled by these glyphs then even though the cursor would appear at the right of the

```
“t”
  -
  a
  M
```

glyphs, the glyph corresponding to the next entered command would appear at the beginning of the following line. Accordingly, the commands “tMt” were entered would result in the display of the following glyphs:

```
  t
  -
  a
  M
t
-
a
```

Thus, in accordance with the invention, the phrase “to the right” includes the situation in which the subsequent glyph appears in the first position on the line below the subsequent glyph. Similarly, a glyph “to the left” of a subsequent glyph can also appear in the last position of the line above the subsequent glyph.

In another embodiment, the second character of a subsequent glyph of the set of the font is positioned above and to the right of the third character of the preceding glyph of the subset. In this embodiment, the glyph of the subset is displayed vertically below the second character of a subsequent glyph of the set of the font, where the subsequent glyph comprises a second character positioned vertically below a first character. Thus, where the glyph of the subset of the font of the invention is displayed vertically below the second character of a subsequent glyph of the set of the font, the glyph of the subset appears to the right of the cursor below a space sufficient to accommodate a subsequent glyph of the set of the font.

In a non-limiting example, the command “t” is assigned to a glyph of the set of the font, namely,

```
“t”,
  -
  a
```

and the command “M” is assigned to the glyph, “M” of the subset of the font that is displayed vertically below the second character of the subsequent glyph. In this example, entry of the command, “t” results in the display of the glyph

```
“t”,
  -
  a
```

where the cursor appears immediately to the right of the glyph. The next command, “M” is assigned to the glyph, “M” of the subset of the font, and is entered immediately after entry of the “t” command. In this example, the commands, “tM” would result in the following glyphs in this non-limiting example of this embodiment of the font of the invention:

t
-
a
M

Continuing this non-limiting example, if the commands “tMt” were entered, the following glyphs are generated:

tt
--
aa
M

It should be noted that in this embodiment of the invention, if the cursor is at the end of a line, where the editing program used allows looping of the glyphs of the font, the glyph of the subset of the font of the invention will appear on the following line below a space sufficient to accommodate a subsequent glyph of the set of the font, and a subsequent glyph of the set of the font will occupy the position directly above the glyph of the subset of the font.

In preferred embodiments, the third character of the glyph represents an amino acid. By “amino acid” is meant any amino acid residue encoded by a three nucleotide codon or any signal to stop encoding an amino acid residue (often depicted as a * symbol or “Ochre,” “Amber,” or “Opal”) encoded by a three nucleotide codon. Ordinarily skilled users of a nucleic acid editing program are aware that a determination of which codons encode which amino acid may be found in the standard genetic code (see, e.g., the genetic code provided in Styer, L., Biochemistry (3^{rd} Edition), W.H. Freeman and Co., New York, 1988). In accordance with the invention, by “protein” or “polypeptide” is meant a chain of two or more amino acid molecules joined with a peptide bond regardless of length or post-translational modification such as acetylation, glycosylation, lipidation, acetylation, or phosphorylation.

Thus, in certain embodiments, the third character (i.e., a glyph of the first subset) is more than one alphabetical letter. For example, the third character may be three alphabetical letters. In this embodiment, the glyph of the subset of the invention need not have the same width as a preceding or subsequent glyph of the set of the font of the invention.

Thus, in one non-limiting example of this embodiment, where the characters of the first subset are all upper case alphabetical characters and the characters of a glyph of a set of the font are lower case alphabetical characters, and where the third character is vertically below a second character, the codon “atg”, which encodes for methionine, may be displayed using the font of the invention by entering “aMtEgT”, which would generate the following glyphs:

atg
---
tac
MET

In an alternate non-limiting example of this embodiment of the invention, the shift key may enable characters displayed by the entered command to appear directly to the left of the next entered command. Thus, the entered commands that display glyphs of the subset of the font display characters having zero-width. Thus, entering “atgMETcccPRO” would generate the following glyphs:

atgccc
------
tacggg
METPRO

In yet another non-limiting example of this embodiment of the invention, the shift key may enable more than one character to be displayed by the entered command. For example, the command “M” may display a glyph “Met” that appears directly below the second character of the preceding glyph of the set of the font. Thus in this example, entering “atgM” would generate the following glyphs:

atg
---
tac
Met

In these examples, a command entered with the shift key (e.g., “M”) displays a glyph of the first subset of the font, while a command entered without the shift key (e.g., “a”) displays a glyph of the set of the font.

In certain embodiments of the font of the invention, the font comprises an additional second subset, which comprises an alphanumerical character positioned above or below the position occupied by a glyph of the set of the font. The font of the invention may also comprise further additional subsets. Note that the commands for each of the additional subsets of glyphs of the font occupies zero width. However, the glyphs to which the zero width commands correspond preferably occupy the same width as a glyph of the set of the font.

For example, the font may include additional subsets such that the restriction endonuclease recognition sites in the nucleic acid sequence represented by the font of the invention are identified. In this example, when a restriction endonuclease recognition site is present in the sequence being represented by the font of the invention, commands are entered for displaying the restriction enzyme site. The user could distinguish commands that entered a glyph of the second subset of the font from the commands displaying a glyph of the set of the font and from commands displaying a glyph of the first subset of the font with an additional entered command. For example, if the user wished to enter a glyph of a second subset of the font which displays the character, “E”, the user may choose to simultaneously enter the shift key, the “e” key, and the “˜” key to display an “E” for a glyph of the second subset of the font. While these commands have zero-width as far as the displayed cursor is concerned, the glyphs of the subfont appear either above or below the glyphs of the set of the font. In some embodiments, the glyphs of the subset of the font appear above and to the left of the cursor, above glyphs of the set of the font, where the glyphs of the subset are positioned horizontally adjacent to one another.

In one non-limiting example of this embodiment of the invention, the glyph of the second subset of the font that represents the restriction endonuclease, EcoRI, is displayed directly above the glyphs of the set of the font representing the EcoRI recognition sequence, gaattc. In this example, the shift and “˜” keys enable characters displayed by the entered command to appear directly to the left of the next entered command. Note that any two commands can be entered; thus, the shift and “˜” keys are merely non-limiting examples of two commands in this example. Thus, in this example, if the commands “gaattc˜E˜c˜o˜R˜I” were entered, the resulting glyphs of the set and the subsets of the invention would be displayed:

EcoRI
gaattc
cttaag

Note that the glyphs displaying “EcoRI” may be positioned directly above the first character of the glyph of the set that represents the nitrogenous base within the recognition sequence that is adjacent to the cleavage site. Thus, in this embodiment, since EcoRI cleaves after the 5′ “g” nitrogenous base of the sequence, the following commands, “g˜E˜c˜o˜R˜Iaattc”, would be entered, resulting in the following displayed glyphs:

```
EcoRI
        gaattc
        cttaag
```

In other embodiments, the glyphs of the second subset of the font appear above and to the left of the cursor, above glyphs of the set of the font, where the glyphs of the second subset are positioned vertically adjacent to one another. In a non-limiting example of this embodiment of the invention, the shift and “˜” keys enable characters displayed by the entered command to appear directly above the next entered command. Thus, if the commands “gaattc˜E˜c˜o˜R˜I” were entered, the resulting glyphs of the set and the subsets of the invention would be displayed:

```
          E
          c
          o
          R
          I
gaattc
cttaag
```

Combining the set, first subset, and second subset of the font of the invention in a non-limiting example, if the commands “gaaEtccF˜E˜c˜o˜R˜I” were entered, the resulting glyphs would be displayed:

```
          E
          c
          o
          R
          I
gaattc
cttaag
    E F
```

In another non-limiting example of this embodiment of the invention, the “˜” key may enable more than one character to be displayed by the entered command. In other words, in this example, typing the “˜” key before a second entered command creates a macro. In this example, the command “˜E” may display a glyph “EcoRI” that appears directly above the first character of the preceding glyph of the set of the font, and the commands “E” and “F” may display glyphs “E” and “F”, respectively, that appear directly below the second character of the preceding glyph of the set of the font. Thus in this example, entering “gaaEtccF˜E” would generate the following glyphs:

```
          E
          c
          o
          R
          I
gaattc
cttaag
    E F
```

In additional embodiments, other command keys displaying glyphs of zero-width as far as the displayed cursor is concerned can be used to enter the number of a particular nitrogenous base in the sequence. Thus, in certain embodiments, the font further comprises a third subset of glyphs, wherein the glyph of third the subset displays an numerical character. For example, if the 5′ “g” of the EcoRI recognition site is the 501th nitrogenous base in the sequence, then command keys displaying these numbers may result in the display of glyphs corresponding to these command keys either above, below, or adjacent to the glyph of the set of the font entering the nucleic acid sequence. According to the various embodiments of the font of the invention, the embodiment allowing the display of the nitrogenous base number may be combined with the embodiment allowing display of the encoded amino acid sequence as well as the embodiments allowing the display of the restriction endonuclease recognition sites.

In one non-limiting example of this combination, the command keys “g501aaEttcF˜E˜c˜o˜R˜I” might result in the following displayed glyphs:

```
              E
              c
              o
              R
501 I
    gaattc
    cttaag
      E F
```

The invention is particularly useful for quickly displaying and editing genetic information that has been modified by standard molecular biology techniques (see, e.g., Ausubel et al., Current Protocols in Molecular Biology, John Wiley and Sons, Inc., New York, N.Y. 1993; Ausubel et al., Short Protocols in Molecular Biology, 4^{th} Ed., John Wiley and Sons, Inc., New York, N.Y. 1999). As described above, when genetic information is displayed using a font (e.g., Courier or Monaco) that is one character in height, displaying and editing modified genetic information (e.g., where the displayed nucleic acid sequence has by modified by, e.g., insertion of a restriction endonuclease recognition sequence) requires repeated cutting and pasting of sequences. The opportunity for error is large, particularly when the sequences to be cut and pasted are longer than one line of text on a page (e.g., where the modified genetic information is a nucleic acid sequence encoding a fusion protein).

For example, one line of text in 12 point Courier font a standard 8.5×11 inch page in portrait format contains approximately 65 characters. If, however, the two sequences to be cut and pasted each consist of more than 65 characters, then the user would have to highlight the sequence to be inserted (the “first sequence”) with his cursor, cut the sequence, locate the position in the second sequence (into which the first sequence is to be inserted), and paste it in. Because a nucleic acid sequence is typically at least two characters in height, repeated cutting and pasting is required to correct for wrapping and the complementary sequences of the second sequence, thus creating opportunity for error. If the two sequences are so large that they occupy more than one 8.5″×11″ page, even the cutting and pasting steps themselves (given the size of the text to be inserted) can be difficult and error-prone.

Using the invention to edit and display modified genetic information, the user can simply highlight the nucleic acid sequence to be inserted, cut that first sequence, and paste it into the second sequence (see, e.g., Examples 3 and 4). Because the font of the invention is more than one character in height, highlighting the sequence allows the user to cut a sequence that is two or more characters in height, and paste that sequence into a second sequence that is also two or more characters in height.

The present invention provides a method for displaying genetic information represented by a set of glyphs, wherein each glyph of the set comprises a first alphanumerical character positioned vertically above a second alphanumerical character, wherein the first alphanumerical character represents a first nitrogenous base and the second alphanumerical character represents a second nitrogenous base that is complementary to the first nitrogenous base. The method comprises receiving an entered command to display one of the set of glyphs; identifying the glyph of the set assigned to the entered command; and displaying the identified glyph.

The invention also provides a computer program product in a computer-readable media for displaying genetic information represented by a set of glyphs, wherein each glyph of the set comprises a first alphanumerical character positioned vertically above a second alphanumerical character, wherein the first alphanumerical character represents a first nitrogenous base and the second alphanumerical character represents a second nitrogenous base that is complementary to the first nitrogenous base. In accordance with the invention, the computer program product comprises means for receiving an entered command to display one of the set of glyphs; means for identifying the glyph of the set assigned to the entered command; and means for displaying the identified glyph.

In addition, the invention provides a computer comprising at least one processor; memory associated with the at least one processor; a display; and a program supported in the memory for displaying genetic information represented by a set of glyphs, wherein each glyph of the set comprises a first alphanumerical character positioned vertically above a second alphanumerical character, wherein the first alphanumerical character represents a first nitrogenous base and the second alphanumerical character represents a second nitrogenous base that is complementary to the first nitrogenous base. According to the invention, the program comprises means for receiving an entered command to display one of the set of glyphs; means for identifying the glyph of the set assigned to the entered command; and means for displaying the identified glyph.

As described above, in some embodiments of the invention, the command is entered using a standard keyboard. In some embodiments, each glyph further comprises a horizontal line between the first alphanumerical character and the second alphanumerical character. In some embodiments, the glyph is displayed on a display screen with a cursor at a location on the screen, the location being the location of the cursor when the command is entered.

In various embodiments, the method, computer program, and computer of the invention are further modified to include further subsets of glyphs.

For example, the method of the invention can further include entering a command assigned to a glyph of a second set, wherein each glyph of the second set is assigned to a single letter or three letter character representing an amino acid residue (see Examples I and II, respectively). Thus, in this embodiment, the invention features a method for displaying a double stranded codon and an amino acid encoded by the codon comprising entering a first command, a second command, a third command, and a fourth command, wherein the first, second, and third commands are each assigned to a glyph comprising a first character positioned vertically above a second character and wherein the fourth command is assigned to a glyph comprising a third character positioned vertically above a first character of a glyph assigned to any one of the first command, the second command, or the third command, or the third character is positioned vertically below a second character of a glyph assigned to any one of the first command, the second command, or the third command.

In another embodiment, the method of the invention further includes entering a command assigned to a glyph of a third and/or fourth set, wherein each glyph of the third set represents a restriction endonuclease, and wherein each glyph of the fourth set represents a position in a sequence (see, e g., Example 3 below).

The invention provides a method for displaying a double-stranded codon and an amino acid encoded by the codon, wherein the method comprises receiving an entered first command, second command, third command, and fourth command, wherein each of the first command, second command, and third command, command is assigned to a glyph of a first set of glyphs, wherein the glyph of the first set comprises a first alphanumerical character positioned vertically above a second alphanumerical character, wherein the first alphanumerical character represents a first nitrogenous base and the second alphanumerical character represents a second nitrogenous base that is complementary to the first nitrogenous base; receiving a fourth command, wherein the fourth command is assigned to a glyph of a second set of glyphs, wherein the glyph of the second set comprises a third alphanumerical character positioned vertically above or below a second character of a glyph displayed by any one of the first command, the second command, or the third command; identifying the glyph assigned to each of the entered first command, second command, third command, and fourth command; and displaying the identified glyphs.

In addition, the invention provides a computer program product in a computer-readable media for displaying a double-stranded codon and an amino acid encoded by the codon, the computer program product comprising means for receiving an entered first command, second command, third command, and fourth command, wherein each of the first command, second command, and third command, command is assigned to a glyph of a first set of glyphs, wherein the glyph of the first set comprises a first alphanumerical character positioned vertically above a second alphanumerical character, wherein the first alphanumerical character represents a first nitrogenous base and the second alphanumerical character represents a second nitrogenous base that is complementary to the first nitrogenous base; means for receiving a fourth command, wherein the fourth command is assigned to a glyph of a second set of glyphs, wherein the glyph of the second set comprises a third alphanumerical character positioned vertically above or below a second character of a glyph displayed by any one of the first command, the second command, or the third command; means for identifying the glyph assigned to each of the entered first command, second command, third command, and fourth command; and means for displaying the identified glyphs.

The invention also provides a computer comprising at least one processor; memory associated with the at least one processor; a display; and a program supported in the memory for displaying genetic information represented by a set of glyphs, wherein each glyph of the set comprises a first alphanumerical character positioned vertically above a second alphanumerical character, wherein the first alphanumerical character represents a first nitrogenous base and the second alphanumerical character represents a second nitrogenous base that is complementary to the first nitrogenous base. In accordance with the invention, the program comprises means for receiving an entered first command, second command, third command, and fourth command, wherein each of the first command, second command, and third command, command is assigned to a glyph of a first set of glyphs, wherein the glyph of the first set comprises a first alphanumerical character positioned vertically above a second alphanumerical character, wherein the first alphanumerical character represents a first nitrogenous base and the second alphanumerical character represents a second nitrogenous base that is complementary to the first nitrogenous base; means for receiving a fourth command, wherein the fourth command is assigned to a glyph of a second set of glyphs, wherein the glyph of the second set comprises a third alphanumerical character positioned vertically above or below a second character of a glyph displayed by any one of the first command, the second command, or the third command; means for identifying the glyph assigned to each of the entered first command, second command, third command, and fourth command; and means for displaying the identified glyphs.

In certain embodiments of the invention, the fourth command is entered after each of the first command, the second command, and the third command is entered. In other embodiments, the fourth command is entered before at least one of the first command, the second command, and the third command is entered.

As used herein, by “codon” is meant three consecutive nitrogenous bases, wherein the three bases encode a single amino acid residue when the bases are translated. The genetic code is well known and is based upon the three bases being read in a 5′ to 3′ direction. Thus, the codon 5′atg3′ encodes a methionine amino acid residue. It should be noted that codons are typically represented as RNA, not DNA nitrogenous bases; however, the ordinarily skilled biologist, bioinformaticist, or chemist would understand that a codon can be represented by DNA nitrogenous bases simply by replacing a uracil (i.e., “U”) nitrogenous base with a thymine (i.e., “T”) nitrogenous base. Of course, if a codon is represented using DNA nitrogenous bases, it is convenient to make the codon double-stranded, where the codon that is translated being on the upper strand, which is represented left to right in the 5′ to 3′ direction. In other words, as used herein, the double stranded codon:

atg
---
tac

encodes methionine and does not encode histidine (which is encoded by the 5′cat3′ codon. Thus, in this example, the entered commands “atgM” (where the “M” command displayed a glyph of the first subset of the font of zero width that appeared directly below the second character of the preceding glyph of the set) would display the glyphs:

atg
---
tac
M

The following examples are intended to further illustrate certain preferred embodiments of the invention and are not limiting in nature.

### EXAMPLE 1

### Generation of a Genetic Font

Using the software application, FontLab (commercially available from Pyrus N.A. Ltd., Box 465, Millersville, Md. 21108, USA), a new font was created. Although in this particular example, the Fontlab application was used, other font generating applications are commercially available including Fontographer (commercially available from Macromedia, Inc., 600 Townsend Street, San Francisco, Calif. 94103, USA), TypeStyler (commercially available from Strider Software, 1605 7th Street Menominee, Mich. 49858-2815, USA).

The newly created font, which was called the Genetics Font, was specifically designed to facilitate the display and editing of nucleic acid molecules. In addition, Genetics Font was designed to also allow the display of the protein sequence encoded to the nucleic acid molecule.

In the Genetics Font, commands entered by typing keystrokes on a standard keyboard were used to specify each glyph. The Genetics Font features a set of glyphs, comprising glyphs assigned to lower case keystrokes, as well as a first subset of glyphs, comprising glyphs assigned to a upper case keystrokes. Where the entered keystroke was lower case, the glyph of the set of the font assigned to the entered keystroke was a first character positioned vertically above a horizontal line which, in turn, was positioned vertically above a second character. The second character represented a nitrogenous base that is complementary to the nitrogenous base represented by the first character.

All of the lower case key strokes were assigned a particular glyph. In the Genetic Font, the lower case keystrokes, and the glyph assigned to that keystroke, were as follows in Table I.

TABLE I

| keystroke | glyph in the Genetics Font |
|---|---|
| a | a |
| | - |
| | t |
| b | b |
| | - |
| | v |
| c | c |
| | - |
| | g |
| d | d |
| | - |
| | h |
| g | g |
| | - |
| | c |
| h | h |
| | - |
| | d |
| m | m |
| | - |
| | k |
| n | n |
| | - |
| | n |
| r | r |
| | - |
| | y |
| s | s |
| | - |
| | s |
| t | t |
| | - |
| | a |
| v | v |
| | - |
| | b |
| w | w |
| | - |
| | w |
| y | y |
| | - |
| | r |

In the Genetics Font of this example, the first and second characters of the glyphs of the set of the font of the invention are as follows on Table II, where the “character” is the first character and the “complementary character” is the second character.

TABLE II

| | | Complementary |
|---|---|---|
| Character | Character Stands for: | Character |
| a | adenine | t |
| b | not adenine (i.e., thymidine, cytosine, or | v |
| | guanine) | |
| c | cytosine | g |
| d | not cytosine (i.e., adenine, thymidine, or | h |
| | guanine) | |
| g | guanine | c |
| h | not guanine (i.e., adenine, thymidine, or | d |
| | cytosine) | |
| k | keto (i.e., guanine or thymidine) | m |
| m | amino (i.e., adenine or cytosine) | k |
| n | adenine, thymidine, cytosine, or guanine | n |
| r | purine (i.e., adenine or guanine) | y |
| s | strong (i.e., cytosine or guanine) | s |
| t | thymidine | a |
| v | not thymidine (i.e., adenine, cytosine, or | b |
| | guanine) | |
| w | weak (i.e., adenine or thymidine) | w |
| y | pyrimidine (i.e., cytosine or thymidine) | r |

In addition, the Genetic Font was designed to include a first subset of glyphs assigned to uppercase keystrokes. Here, the entered uppercase keystroke was assigned to a glyph displayed below a glyph of the set displayed by a lower case keystroke. In the first of two variations, the lower case keystroke (displaying a glyph of the set of the font) may have already been entered, in which case the glyph of the first subset displayed by an entered upper case keystroke is displayed below the glyph of the set appearing at the immediate left of the cursor. In a second variation, the lower case keystroke has not yet been entered, in which case the glyph of the subset of the font displayed by the entered upper case keystroke is displayed below the space appearing at the immediate right of the cursor, where the space can accommodate a glyph of the set displayed by a subsequently entered lower case keystroke.

In this example, where the third character of the first subset of the glyph is an amino acid, the upper case keystrokes and assigned third characters of the glyph of the subset of the font of the invention are as follows on Table III.

TABLE III

| Keystroke Command | Third Character | Stands for: |
|---|---|---|
| A | A | alanine |
| B | B | asparagine or aspartic acid |
| C | C | cysteine |
| D | D | aspartic acid |
| E | E | glutamic acid |
| F | F | phenylalanine |
| G | G | glycine |
| H | H | histidine |
| I | I | isoleucine |
| K | K | lysine |
| L | L | leucine |
| M | M | methionine |
| N | N | asparagine |
| P | P | proline |
| Q | Q | glutamine |
| R | R | arginine |
| S | S | serine |
| T | T | threonine |
| V | V | valine |
| W | W | tryptophan |
| X | X | any amino acid |
| Y | Y | tyrosine |
| Z | Z | glutamine or glutamic acid |
| * | * | stop signal |

The Genetic Font was constructed for use by the ordinarily skilled biologist or bioinformaticist who would understand that any amino acid or stop signal is necessarily encoded by a codon (i.e., three consecutive nitrogenous bases).

By using the Genetic Font as described in this example, where the glyph of the subset is displayed below the second character of a preceding glyph of the set of the font, the genetic information:

GCTCCTAGTCCAGACGCCATGGGT
————————————————————————
CGAGGATCAGGTCTGCGGTACCCA
A P S P D A M G

is displayed by entering the commands, “gActcPctaSgtcPcagDacgAccaMtggGggt”.

Use of the version of the Genetic Font of this example where the glyph of the subset is displayed below the second character of a subsequent glyph of the set of the font, the above genetic information is displayed by making the keystrokes, “AgctPcctSagtPccaDgacAgccMatgGggt”.

### EXAMPLE 2

### Generation of a Three-Code Amino Acid Genetics Font

In a variation of the Genetics Font described in Example 1, a Three-Code Amino Acid Genetic Font is generated. The set of glyphs in the font that are assigned to lower case keystrokes are the same as in the Genetics Font of Example 1; however, the first subset of glyphs assigned to an upper case keystroke comprise a third character, wherein the third character is three consecutive alphabetical letters. In the Three-Code Amino Acid Genetics Font, the glyph of the first subset of the invention does not necessarily have the same width as the set of the Three-Code Amino Acid Genetics Font under which it appears.

In this example, the upper case keystrokes that display third characters of the glyph of the first subset of the font of the invention are as follows on Table IV.

TABLE IV

| Keystroke Command | Character | Stands for: |
|---|---|---|
| A | Ala | alanine |
| B | Asx | asparagine or aspartic acid |
| C | Cys | cysteine |
| D | Asp | aspartic acid |
| E | Glu | glutamic acid |
| F | Phe | phenylalanine |
| G | Gly | glycine |
| H | His | histidine |
| I | Ile | isoleucine |
| K | Lys | lysine |
| L | Leu | leucine |
| M | Met | methionine |
| N | Asn | asparagine |
| P | Pro | proline |
| Q | Gln | glutamine |
| R | Arg | arginine |
| S | Ser | serine |
| T | Thr | threonine |
| V | Val | valine |
| W | Trp | tryptophan |
| X | Xaa | any amino acid |
| Y | Tyr | tyrosine |
| Z | Glx | glutamine or glutamic acid |

By using the Three-Code Amino Acid Genetics Font as described in this example, where the glyph of the subset is displayed below a preceding glyph of the set of the font, the genetic information,

GCTCCTAGTCCAGACGCCATGGGT
————————————————————————
CGAGGATCAGGTCTGCGGTACCCA
AlaProSerProAspAlaMetGly

is displayed by entering the keystrokes, “gActcPctaSgtcPcagDacgAccaMtggGggt”.

Use of the version of the Three-Code Amino Acid Genetics Font of this example where the glyph of the first subset is displayed below a subsequent glyph of the set of the font, the above-sequence is displayed by making the keystrokes, “AgctPcctSagtPccaDgacAgccMatgGggt”.

### EXAMPLE 3

### Editing a Sequence Written in the Genetic Font

In this example, the Genetics Fonts of Example I is used to add a nucleic acid sequence encoding a histidine tag (“his tag”) to a nucleic acid sequence encoding a protein to facilitate the purification of the encoded his tagged protein using a substrate which binds to the histidine tag (e.g., nickel-N-(5-amino-1-carboxypentyl)iminodiacetic acid (NTA) (“Ni-NTA”). (Ni-NTA agarose is commercially available from, for example, from QIAGEN Inc., Valencia, Calif.) A standard histidine tag comprises 6 to 8 histidine residues. Since histidine is encoded by codons 5′ cac 3′ or 5′ cag 3′, in this example, the ribonucleic acid sequence encoding the his tag has the following RNA sequence:

5′ caccagcaccagcaccagcaccag 3′

Double-stranded codons encoding the his tag and a 3′ terminal stop codon (encoding a stop signal) has the following DNA and amino acid sequence:

caccagcaccagcaccagcaccagtga
———————————————————————————
gtggtcgtggtcgtggtcgtggtcact
H H H H H H H H *

Using the font of the invention, a nucleic acid sequence encoding a histidine tag is added to the 3′ end of the following nucleic acid sequence encoding the indicated polypeptide:

GCTCCTAGTCCAGACGCCATGGGTCATTTCACAGCCCGGGACAAGGCTACTATCACAAGC
————————————————————————————————————————————————————————————
CGAGGATCAGGTCTGCGGTACCCAGTAAAGTGTCGGGCCCTGTTCCGATGATAGTGTTCG
A P S P D A M G H F T P G D K A T I T S

by simply placing highlighting the glyphs desired starting with the

```
“ c”
    —
    g
    H
```

glyph and dragging the cursor to the right until the entire nucleic acid and amino acid sequence encoding the his tag and stop signal are highlighted. The user can then copy the highlighted text and, placing his cursor to the right of the above nucleic acid sequence, paste in the highlighted text.

By using the font of the invention, the resulting genetic information will be displayed:

GCTCCTAGTCCAGACGCCATGGGTCATTTCACAGCCCGGGACAAGGCTACTATCACAAGC caccagc
———————————————————————————————————————————————————————————— ———————
CGAGGATCAGGTCTGCGGTACCCAGTAAAGTGTCGGGCCCTGTTCCGATGATAGTGTTCG gtggtcg
A P S P D A M G H F T P G D K A T I T S H H H
accagcaccagcaccagtga
————————————————————
tggtcgtggtcgtggtcact
H H H H H *

Note that there may or may not be a word wrap function in a font of the invention. In this example, there is a word wrap function; thus, when the nucleic acid sequence encoding the his tag and stop signal are pasted onto the nucleic acid sequence encoding the above polypeptide, the pasted sequence wraps onto the next line.

For illustrative purposes, if the nucleic acid sequence encoding the his tag and stop signal is to be pasted onto the above sequence, but the font of the invention is not being used, cutting and pasting as described above would result in the following being displayed:

GCTCCTAGTCCAGACGCCATGGGTCATTTCACAGCCCGGGACAAGGCTACTATCACAAGC
caccagcaccagcaccagcaccagtga
———————————————————————————
gtggtcgtggtcgtggtcgtggtcact
H H H H H H H H *
————————————————————————————————————————————————————————————
CGAGGATCAGGTCTGCGGTACCCAGTAAAGTGTCGGGCCCTGTTCCGATGATAGTGTTCG
A P S P D A M G H F T P G D K A T I T S

The user would then have to cut and paste numerous times to obtain a double-stranded nucleic acid sequence and encoded amino acid sequence. As discussed earlier, repeated cutting and pasting can lead to inadvertent errors in the sequence.

### EXAMPLE 4

### Editing a Sequence Written in the Genetic Font

In this example, the Genetics Fonts of Example I is used to add a nucleic acid sequence encoding BamHI restriction endonuclease recognition site (“BamHI site”) into the middle of a nucleic acid sequence.

BamHI recognizes the following DNA sequence:

5′ GGATCC 3′
CCTAGG

Using the font of the invention, a nucleic acid sequence encoding a BamHI site is inserted into the area (indicated with the arrow) of the promoter region of the human Siah-1 gene (Maeda et al., FEBS Lett. 512 (1-3), 223-226, 2002) indicated by the arrow in the following sequence:

↓
acaagttggggacctgctttcctttgcaaa
tgttcaacccctggacgaaaggaaacgttt

Using the font of the invention, the six glyphs:

“GGATCC”,
CCTAGG

which are used to create the BamHI site are simply highlighted using the cursor, and cut. Next, the cursor is placed at the position in the human Siah-1 gene (i.e., between the “c” and “t” glyphs, and the BamHI glyphs are pasted in.

By using the font of the invention, the resulting genetic information will be displayed:

acaagttggggacctgcGGATCCtttcctttgcaaa
tgttcaacccctggacgCCTAGGaaaggaaacgttt

For illustrative purposes, if the BamHI sequence is to be pasted onto the above sequence, but the font of the invention is not used, cutting and pasting as described above would result in the following being displayed:

```
acaagttggggacctgcGGATCC
                                  CCTAGGtttcctttgcaaa
          tgttcaacccctggacgaaaggaaacgttt
```

The user would then have to cut the “CCTAGG” sequences out of the above sequence, carefully delete the spaces (without deleting any characters) such that the “tttcctttgcaaa” sequence would exactly follow the “GGATCC” sequence, then align the “tgttcaacccctggacgaaaggaaacgttt” sequence below the upper sequence such that the left-most “t” character is directly below the “a” character. Next, the user would need to past the “CCTAGG” sequence in between the correct “g” and “a” characters of the bottom sequence such that all characters aligned with their complementary characters. Only by accurately cutting and pasting each of these sequences, would the user be able to obtain a double-stranded nucleic acid sequence containing a BamHI site in its midst. While this accurate cutting and pasting may be readily performed for insertion of a single nucleic acid sequence, with repeated insertions of nucleic acid sequences, the opportunity for error is multiplied.

Moreover, for illustration purposes, the BamHI site in this example is been displayed with capitalized alphanumerical characters while the characters of the human Siah-1 gene is displayed with lowercase alphanumerical characters. In practice, all of the character are likely to be either lower or upper case. Given the numerous cutting and pasting steps, not to mention the steps needed to delete the spaces between the sequences, ample opportunity for inadvertent errors exists, leading to errors in the final sequence.

### EQUIVALENTS

As will be apparent to those skilled in the art to which the invention pertains, the present invention may be embodied in forms other than those specifically disclosed above without departing from the spirit or essential characteristics of the invention. For example, those skilled in the art will recognize, or be able to ascertain, using no more than routine experimentation, numerous equivalents to the specific substances and procedures described herein. The particular embodiments of the invention described above, are, therefore, to be considered as illustrative and not restrictive. The scope of the invention is as set forth in the appended claims rather than being limited to the examples contained in the foregoing description.

The published patent and scientific literature referred to herein establishes knowledge that is available to those with skill in the art. The literature references, including GenBank database sequences, that are cited herein are hereby incorporated by reference to the same extent as if each was specifically and individually indicated to be incorporated by reference.

1. A method for displaying genetic information represented by a set of glyphs, wherein each glyph of the set comprises a first alphanumerical character positioned vertically above a second alphanumerical character, wherein the first alphanumerical character represents a first nitrogenous base and the second alphanumerical character represents a second nitrogenous base that is complementary to the first nitrogenous base, the method comprising:
a) receiving an entered command to display one of the set of glyphs, wherein the command is entered using a standard keyboard;
b) identifying the glyph of the set assigned to the entered command; and
c) displaying the identified glyph, wherein the glyph is displayed at a location on a display screen with a cursor, the location of the displayed glyph being the location of the cursor when the command is entered.

2. The method of claim 1, wherein each glyph of the set further comprises a horizontal line between the first alphanumerical character and the second alphanumerical character.

3. The method of claim 1, wherein the genetic information is further represented by a subset of glyphs comprising a third alphanumerical character, the method further comprising:
d) receiving an entered command to display one of the subset of glyphs, wherein the command is entered using a standard keyboard;
e) identifying the glyph of the subset assigned to the entered command; and
f) displaying the identified glyph of the subset, wherein the glyph of the subset is displayed at a location on a display screen with a cursor, the location of the displayed glyph of the subset being below the location of the cursor when the command is entered.

4. The method of claim 3, wherein the third alphanumerical character represents an amino acid or a stop signal.

5. The method of claim 3, wherein the location of the displayed glyph of the subset is directly below the previously displayed glyph of the set.

6. The method of claim 3, wherein the location of the displayed glyph of the subset is diagonally below to the right of the previously displayed glyph of the set.

7. The method of claim 1, wherein the genetic information is further represented by a second subset of glyphs comprising a fourth alphanumerical character, the method further comprising:
d) receiving an entered command to display one of the second subset of glyphs, wherein the command is entered using a standard keyboard;
e) identifying the glyph of the second subset assigned to the entered command; and
f) displaying the identified glyph of the second subset, wherein the glyph of the subset is displayed at a location on a display screen with a cursor, the location of the displayed glyph of the second subset being above the location of the cursor when the command is entered.

8. The method of claim 7, wherein the third alphanumerical character represents a restriction endonuclease.

9. The method of claim 7, wherein the location of the displayed glyph of the subset is directly above the previously displayed glyph of the set.

10. A computer program product in a computer-readable media for displaying genetic information represented by a set of glyphs, wherein each glyph of the set comprises a first alphanumerical character positioned vertically above a second alphanumerical character, wherein the first alphanumerical character rep resents a first nitrogenous base and the second alphanumerical character represents a second nitrogenous base that is complementary to the first nitrogenous base, the computer program product comprising:
a) means for receiving an entered command to display one of the set of glyphs;
b) means for identifying the glyph of the set assigned to the entered command; and
c) means for displaying the identified glyph.

11. A computer comprising at least one processor; memory associated with the at least one processor; a display; and a program supported in the memory for displaying genetic information represented by a set of glyphs, wherein each glyph of the set comprises a first alphanumerical character positioned vertically above a second alphanumerical character, wherein the first alphanumerical character represents a first nitrogenous base and the second alphanumerical character represents a second nitrogenous base that is complementary to the first nitrogenous base, the program comprising:
a) means for receiving an entered command to display one of the set of glyphs;
b) means for identifying the glyph of the set assigned to the entered command; and
c) means for displaying the identified glyph.

12. A method for displaying a double-stranded codon and an amino acid encoded by the codon, the method comprising:
a) receiving an entered first command, second command, and third command, wherein each of the first command, second command, and third command, command is assigned to a glyph of a set of glyphs, wherein the glyph of the set comprises a first alphanumerical character positioned vertically above a second alphanumerical character, wherein the first alphanumerical character represents a first nitrogenous base and the second alphanumerical character represents a second nitrogenous base that is complementary to the first nitrogenous base;
b) receiving a fourth command, wherein the fourth command is assigned to a glyph of a subset of glyphs, wherein the glyph of the subset comprises a third alphanumerical character positioned vertically above or below a glyph displayed by any one of the first command, the second command, or the third command;
c) identifying the glyph assigned to each of the entered first command, second command, third command, and fourth command; and
d) displaying the identified glyphs.

13. The method of claim 12, wherein the third alphanumerical character represents an amino acid or a stop signal.

14. A computer program product in a computer-readable media for displaying a double-stranded codon and an amino acid encoded by the codon, the computer program product comprising:
a) means for receiving an entered first command, second command, and third command, wherein each of the first command, second command, and third command, command is assigned to a glyph of a set of glyphs, wherein the glyph of the set comprises a first alphanumerical character positioned vertically above a second alphanumerical character, wherein the first alphanumerical character represents a first nitrogenous base and the second alphanumerical character represents a second nitrogenous base that is complementary to the first nitrogenous base;
b) means for receiving a fourth command, wherein the fourth command is assigned to a glyph of a subset of glyphs, wherein the glyph of the subset comprises a third alphanumerical character positioned vertically above or below a glyph displayed by any one of the first command, the second command, or the third command;
c) means for identifying the glyph assigned to each of the entered first command, second command, third command, and fourth command; and
d) means for displaying the identified glyphs.

15. The method of claim 14, wherein the third alphanumerical character represents an amino acid or a stop signal.

16. A computer comprising at least one processor; memory associated with the at least one processor; a display; and a program supported in the memory for displaying a double-stranded codon and an amino acid encoded by the codon, the program comprising:
a) means for receiving an entered first command, second command, and third command, wherein each of the first command, second command, and third command, command is assigned to a glyph of a set of glyphs, wherein the glyph of the set comprises a first alphanumerical character positioned vertically above a second alphanumerical character, wherein the first alphanumerical character represents a first nitrogenous base and the second alphanumerical character represents a second nitrogenous base that is complementary to the first nitrogenous base;
b) means for receiving a fourth command, wherein the fourth command is assigned to a glyph of a subset of glyphs, wherein the glyph of the subset comprises a third alphanumerical character positioned vertically above or below a glyph displayed by any one of the first command, the second command, or the third command;
c) means for identifying the glyph assigned to each of the entered first command, second command, third command, and fourth command; and
d) means for displaying the identified glyphs.

17. The method of claim 16, wherein the third alphanumerical character represents an amino acid or a stop signal.

18. A method for displaying genetic information in a computer system, the computer system including a monitor and a keyboard, the method comprising:
a) defining a plurality of glyphs, each glyph including at least a first character and a second character, the first and second characters in each of the glyphs representing first and second complementary nitrogenous bases, respectively;
b) defining a plurality of commands that may be entered into the computer system, each command being entered into the computer system by one or more keystrokes of the keyboard;
c) establishing a correspondence between the commands and the glyphs, each of the commands corresponding to one of the glyphs, each command being associated with a first number and a second number, the first number being the number of keystrokes used to enter the command into the computer system, the second number being the number of characters in the glyph corresponding to the command, the second number being greater than the first number; and
d) in response to a first one of the commands being entered into the computer system, displaying the glyph corresponding to the first command on the computer monitor.

19. The method of claim 18, further comprising, in response to a second one of the commands being entered into the computer system, displaying the glyph corresponding to the second command on the computer monitor to the right of and adjacent to the glyph corresponding to the first command.

20. A method for displaying information in a computer system, the computer system including a monitor and a keyboard, the method comprising:
a) displaying two or more adjacent glyphs on the monitor, each glyph including at least a first character and a second character, the first and second characters in each of the glyphs representing first and second complementary nitrogenous bases, respectively; and
b) defining a select command that may be entered into the computer system, the select command permitting simultaneous selection of all characters in one of the displayed adjacent glyphs without also selecting characters in any other of the displayed adjacent glyphs.

21. The method of claim 20, the select command further permitting selection of two or more adjacent glyphs displayed on the monitor.

22. The method of claim 21, further comprising defining a delete command that may be entered into the computer system, the delete command removing all selected glyphs from the monitor.

23. The method of claim 22, a left glyph being a previously displayed glyph to the left of and adjacent to the selected glyphs, a right glyph being a previously displayed glyph to the right of and adjacent to the selected glyphs, the delete command further comprising moving the right glyph to the left so that it is adjacent to the left glyph.

24. The method of claim 23, a right group of glyphs including the right glyph and all previously displayed glyphs to the right of the right glyph, the delete command further comprising moving the right group of glyphs to the left.

25. The method of claim 20, further comprising defining a copy command that may be entered into the computer system, the copy command copying the selected glyphs into a buffer.

26. The method of claim 25, further defining a paste command that may be entered into the computer system, a left end glyph being the glyph at a left end of the selected glyphs, a right end glyph being the glyph at a right end of the selected glyphs, a right glyph being a previously displayed glyph, a left glyph being a previously displayed glyph to the left of and adjacent to the right glyph, the paste command displaying the glyphs in the buffer on the monitor such that the right end glyph is to the left of and adjacent to the right glyph, and such that the left end glyph is to the right of and adjacent to the left glyph.

27. A method for displaying information in a computer system, the computer system including a monitor and a keyboard, the method comprising:
a) defining a plurality of glyphs, each glyph including at least a first character and a second character, the first and second characters in each of the glyphs representing first and second complementary nitrogenous bases, respectively;
b) defining a plurality of commands that may be entered into the computer system, each command being entered into the computer system by applying one or more keystrokes to the keyboard;
c) establishing a correspondence between the commands and the glyphs, each of the commands corresponding to one of the glyphs;
d) establishing a present cursor location on the monitor;
e) in response to one of the commands being entered into the computer system, displaying the glyph corresponding to that command on the monitor at the present cursor location and then moving the present cursor location to the right of the glyph corresponding to that command;
f) repeating step (e) in response to additional commands being entered into the computer; and
g) defining a select command that may be entered into the computer system, the select command permitting simultaneous selection of all characters in a single glyph without also selecting characters in any other displayed glyphs adjacent to the single glyph.
